(19) United States
(12) Patent Application Publication
(10) Pub. No.: US 2007/0173547 A1
(43) Pub. Date: Jul. 26, 2007

(54) Method for producing dry powders of one or several carotenoids

(75) Inventor: Jesper Feldthusen Jensen, Mainz (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/587,562

(22) PCT Filed: Feb. 2, 2005
(86) PCT No.: PCT/EP05/01016

§ 371 Date: Jul. 31, 2006

(30) Foreign Application Priority Data
Feb. 6, 2004 (DE) 10 2004 006 106.8

(51) Int. Cl. A61K 31/22; A61K 31/12; A61K 31/015

(52) U.S. Cl. 514/546; 514/690; 514/763; 264/5

(57) ABSTRACT

The invention relates to a method for producing dry powders of one or several carotenoids, said method being characterised in that a) one or several carotenoids are suspended in an aqueous molecularly dispersed or colloidally dispersed solution of a mixture consisting of trehalose and at least one protein-containing protective colloid and b) the obtained suspension is transformed into a dry powder by separation of water and, optionally, additionally used solvents and by subsequent drying, optionally in the presence of a coating material.

The invention relates to a process for producing dry powders of one or more carotenoids, preferably of xanthophyll-containing dry powders, in particular of xanthophylls selected from the group consisting of astaxanthin, canthaxanthin, lutein, zeaxanthin, citranaxanthin and ethyl β-apo-8′-carotenoate.

The carotenoid class of substances is classified into two main groups, the carotenes and the xanthophylls. The carotenes, which are pure polyene hydrocarbons such as, for example, β-carotene or lycopene, differ from the xanthophylls which also have oxygen functionalities such as hydroxyl, epoxy and/or carbonyl groups. Typical representatives of the latter group are, inter alia, astaxanthin, canthaxanthin, lutein and zeaxanthin.

The oxygen-containing carotenoids also include citranaxanthin and ethyl β-apo-8′-carotenoate.

Oxygen-containing carotenoids are widespread in nature and occur inter alia in corn (zeaxanthin), in green beans (lutein), in paprika (capsanthin), in egg yolk (lutein) and in shrimps and salmon (astaxanthin), conferring on these foodstuffs their characteristic color.

These polyenes, which can both be obtained by synthesis and be isolated from natural sources, represent important coloring materials for the human food and animal feed industries and for the pharmaceutical sector and are, as in the case of astaxanthin, active substances with provitamin A activity in salmon.

Both carotenes and xanthophylls are insoluble in water, while the solubility in fats and oils is found to be only low, however. This limited solubility and the great sensitivity to oxidation stand in the way of direct use of the relatively coarse-particled products obtained by chemical synthesis in the coloring of human foods and animal feeds because, in coarsely crystalline form, the substances are not stable during storage and provide only poor coloring results. These effects which are disadvantageous for use of xanthophylls in practice are particularly evident in an aqueous medium.

Improved color yields in the direct coloring of human foods can be achieved only by specifically produced formulations in which the active substances are in finely divided form and, if appropriate, protected from oxidation by protective colloids. In addition, use of these formulations in animal feeds leads to a greater bioavailability of the carotenoids or xanthophylls and thus indirectly to improved coloring effects, for example in egg yolk or fish pigmentation.

Various processes have been described for improving the color yields and for increasing the absorbability or bioavailability and all of them aim at reducing the size of the crystallites of the active substances and bringing the particles to a size in the region below 10 μm. Numerous methods, inter alia described in Chimia 21, 329 (1967), WO 91/06292 and WO 94/19411, involve the grinding of carotenoids using a colloid mill and thus achieve particle sizes of from 2 to 10 μm.

There also exist a number of combined emulsification/spray drying processes as described, for example, in DE-A-12 11 911 or in Eβ-A-0 410 236.

According to European patent Eβ-B-0 065 193, carotenoid products in finely divided powder form are produced by dissolving a carotenoid in a volatile, water-miscible organic solvent at elevated temperatures, if appropriate under elevated pressure, and precipitating the carotenoid by mixing with an aqueous solution of a protective colloid and then spray drying.

An analogous process for producing carotenoid products in finely divided powder form is described in Eβ-A-0 937 412 with use of water-immiscible solvents.

The nanoparticulate dispersions of xanthophyll active substances produced as described in Eβ-B-0 065 193 frequently display the following phenomena, however.

The aqueous, xanthophyll-containing active substance dispersions are frequently colloidally unstable, especially on concentration. Flocculation of active substance particles, partly by sedimentation and partly by creaming, makes subsequent conversion of the dispersion into a dry powder impossible.

Thus, the great demands on xanthophyll-containing formulations in relation to coloring effect and bioavailability cannot always be met because of the problems described with the abovementioned process.

Another disadvantage of gelatins is that they have strongly adhesive properties. With the drying methods customary for liquid systems, such as, for example, spray drying, on use of gelatin-containing products there may be thread formation or caking.

An additional factor is the diminishing acceptance of gelatin-containing products by consumers.

In other protective colloids which are often used, such as gum arabic, starch, dextrins, pectin or tragacanth, it is frequently possible to incorporate only relatively low concentrations of lipid-soluble substances. In addition gum arabic in particular has in the past not always been available in sufficient quality because of poor harvests.

Synthetic colloids such as polyvinylpyrrolidone or semisynthetic polymers such as cellulose derivatives likewise show a limited emulsifying capacity and are not always accepted, especially in the human foods sector.

DE-A-44 24 085 describes the use of partially degraded soybean proteins as protective colloids for lipid-soluble active substances. The soybean proteins disclosed herein have a degree of hydrolysis of from 0.1 to 5%. The color strength of the formulations produced with these protective colloids is not always satisfactory.

German published specification DE-A-101 04 494 describes the production of carotenoid dry powders by using soybean proteins together with lactose as protective colloids. Despite improved cold water redispersibility and increased coloring strength of the carotenoid preparations disclosed herein, the stability during storage of these formulations, especially when the active substance content is high, is not always satisfactory.

It is an object of the present invention to propose processes for producing carotenoid-containing dry powders, in particular dry powders of oxygen-containing carotenoids, which do not display the abovementioned disadvantages of the prior art and which enable a high carotenoid content to be achieved in the preparation.

We have found that this object is achieved by a process for producing dry powders of one or more carotenoids, which comprises
a) suspending one or more carotenoids in an aqueous molecular or colloidal solution of a mixture of trehalose and at least one protein-containing protective colloid and
b) converting the suspension which has formed into a dry powder by removing the water and, if appropriate, additionally used solvents and subsequent drying, if appropriate in the presence of a coating material.

Suitable protein-containing protective colloids are:

gelatin, for example pig or fish gelatin, in particular acid- or base-degraded gelatin having Bloom numbers in the range from 0 to 250, very particularly preferably gelatin A 100 and A 200, and low molecular weight, enzymatically degraded gelatin types having the Bloom number 0 and molecular weights of from 15 000 to 25 000 D, such as, for example, Collagel A and Gelitasol P (from Stoess, Eberbach) and mixtures of these gelatin types;

caseine and/or a caseinate, for example sodium caseinate;

vegetable proteins such as soybean, rice and/or wheat proteins, it being possible for these vegetable proteins to be in partially degraded or in non-degraded form.

Preferred protective colloids used for the purposes of the present invention are caseine or a caseinate or mixtures thereof. Sodium caseinate should be mentioned as particularly preferred protective colloid.

A preferred embodiment of the abovementioned process comprises grinding the suspension prepared in process step a) before conversion into a dry powder. In this case, the active substance [the carotenoid(s)] is preferably suspended in crystalline form before the grinding process.

The grinding can take place in a manner known per se, for example using a ball mill. This entails, depending on the type of mill used, grinding until the particles have an average particle size D[4.3] determined by Fraunhofer diffraction of from 0.1 to 100 μm, preferably 0.2 to 50 μm, particularly preferably 0.2 to 20 μm, very particularly preferably 0.2 to 5 μm, especially 0.2 to 0.8 μm. The term D[4.3] refers to the volume-weighted average diameter (see Handbook for Malvern Mastersizer S, Malvern Instruments Ltd., UK).

Further details of the grinding and the apparatus employed therefor are to be found, inter alia, in Ullmann's Encyclopedia of Industrial Chemistry, Sixth Edition, 2000, Electronic Release, Size Reduction, Chapter 3.6.: Wet Grinding, and in Eβ-A-0 498 824.

A likewise preferred variant of the process of the invention comprises the suspension in stage a) comprising the following steps:
a₁) dissolving one or more carotenoids in a water-miscible organic solvent or in a mixture of water and a water-miscible organic solvent or
a₂) dissolving one or more carotenoids in a water-immiscible organic solvent and
a₃) mixing the solution obtained as in a,) or a₂) with an aqueous molecular or colloidal solution of a mixture of trehalose and at least one protein-containing protective colloid, resulting in the hydrophobic phase of the carotenoid as nanodisperse phase.

The water-miscible solvents used in stage a₁) are, in particular, water-miscible, thermally stable, volatile solvents comprising only carbon, hydrogen and oxygen, such as alcohols, ethers, esters, ketones and acetals. The solvents expediently used are those which are at least 10% water-miscible, have a boiling point below 200° C. and/or have fewer than 10 carbons. Those particularly preferably used are methanol, ethanol, n-propanol, isopropanol, 1,2-butanediol 1-methyl ether, 1,2-propanediol 1-n-propyl ether, tetrahydrofuran or acetone.

The term “a water-immiscible organic solvent” means for the purpose of the present invention an organic solvent with a solubility in water of less than 10% under atmospheric pressure. Possible solvents in this connection are, inter alia, halogenated aliphatic hydrocarbons such as, for example, methylene chloride, chloroform and tetrachloromethane, carboxylic esters such as dimethyl carbonate, diethyl carbonate, propylene carbonate, ethyl formate, methyl, ethyl or isopropyl acetate and ethers such as methyl tert-butyl ether. Preferred water-immiscible organic solvents are the following compounds from the group consisting of dimethyl carbonate, propylene carbonate, ethyl formate, ethyl acetate, isopropyl acetate-and methyl tert-butyl ether.

The process of the invention preferably involves the production of dry powders of oxygen-containing carotenoids, particularly preferably compounds selected from the group consisting of astaxanthin, canthaxanthin, lutein, zeaxanthin, citranaxanthin and ethyl β-apo-8′-carotenoate, very particularly preferably astaxanthin and canthaxanthin.

The abovementioned dry powders are advantageously produced in such a way that at least one of the carotenoids is dissolved in a water-miscible organic solvent at temperatures above 30° C., preferably between 50° C. and 240° C., in particular 100° C. to 200° C., particularly preferably 1400C to 180° C., if appropriate under pressure.

Since exposure to high temperatures may in some circumstances reduce the desired high proportion of all-trans isomer, the dissolving of the carotenoid(s) takes place as quickly as possible, for example in the region of seconds, e.g. in 0.1 to 10 seconds, particularly preferably in less than 1 second. For rapid preparation of the molecular solution it may be advantageous to apply elevated pressure, e.g. in the range from 20 bar to 80 bar, preferably 30 to 60 bar.

To the molecular solution obtained in this way is subsequently added directly the aqueous molecular or colloidal solution, which is cooled if appropriate, of the mixture of trechalose and at least one protein-containing protective colloid in such a way that a mixing temperature of about 35° C. to 80° C. is set up.

During this, the solvent component is transferred into the aqueous phase, and the hydrophobic phase of the carotenoid(s) results as nanodisperse phase.

Reference is made at this point to EP B-0 065 193 for a detailed description of the process and apparatus for the abovementioned dispersion.

The invention likewise relates to a process for producing an astaxanthin dry powder, wherein
a) astaxanthin is dissolved in a water-miscible organic solvent or a mixture of water and a water-miscible organic solvent at temperatures above 30° C.,
b) the resulting solution is mixed with an aqueous molecular or colloidal solution of a mixture of trehalose with casein or a caseinate or a mixture of trehalose with casein and a caseinate, and
c) the suspension which has formed is converted into a dry powder.

A process for producing astaxanthin-containing dry powders using a mixture of trehalose and casein and/or sodium caseinate, in particular of trehalose and sodium caseinate, is very particularly preferred in this connection.

The conversion into a dry powder can take place inter alia by spray drying, spray cooling, freeze drying or drying in a fluidized bed, if appropriate also in the presence of a coating material. Suitable coating agents are, inter alia, corn starch, silica or else tricalcium phosphate.

To increase the stability of the active substance to oxidative degradation it is advantageous to add stabilizers such as α-tocopherol, t-butylhydroxytoluene, t-butylhydroxyanisole, ascorbic acid, sodium ascorbate or ethoxyquin in a concentration of from 2 to 10% by weight, preferably 3 to 7% by weight, based on the dry mass of the powder. They can be added either to the aqueous or to the solvent phase, but they are preferably dissolved together with the active substances in the solvent phase.

To increase the stability of the active substance to microbial degradation, it may be expedient to add preservatives such as, for example, methyl 4-hydroxybenzoate, propyl 4-hydroxybenzoate, sorbic acid or benzoic acid or their salts to the preparation.

It may also be advantageous in some circumstances additionally for a physiologically acceptable oil such as, for example, sesame oil, corn oil, cottonseed oil, soybean oil or peanut oil, and esters of medium chain-length vegetable fatty acids, in a concentration of from 0 to 500% by weight, preferably 10 to 300% by weight, particularly preferably 20 to 100% by weight, based on the xanthophyll(s), to be dissolved in the solvent phase and then precipitated as extremely fine particles together with the active substances and said additives on mixing with the aqueous phase.

The ratio of protective colloid and trehalose to carotenoid is generally chosen so that the resulting final product comprises from 0.1 to 40% by weight, preferably 1 to 35% by weight, particularly preferably 5 to 30% by weight, very particularly preferably 10 to 25% by weight of at least one carotenoid, 1 to 40% by weight, preferably 2 to 30% by weight, particularly preferably 3 to 20% by weight, very particularly preferably 5 to 15% by weight of at least one protective colloid and 10 to 80% by weight, preferably 20 to 75% by weight, particularly preferably 30 to 70% by weight, very particularly preferably 40 to 60% by weight of trehalose, all percentages based on the dry mass of the powder, and, if appropriate, small amounts of stabilizers and preservatives.

The invention also relates to dry powders of carotenoids obtainable by one of the processes mentioned at the outset.

These are preferably dry powders comprising oxygen-containing carotenoids selected from the group consisting of astaxanthin, canthaxanthin, lutein, zeaxanthin, citranaxanthin and ethyl β-apo-8′-carotenoate, particularly preferably canthaxanthin and astaxanthin, very particularly preferably astaxanthin.

The content of astaxanthin in the preparations of the invention is preferably in the range from 10 to 25% by weight.

The dry powders of the invention are distinguished inter alia by the fact that they can be redispersed without problems in aqueous systems to result in a uniform fine distribution of the active substance in the particle size range below 1 μm.

The use of a combination of trehalose and protein-containing protective colloids, in particular casein or sodium caseinate, as formulation excipients has the advantage compared with other sugars, for example lactose or sucrose, that the carotenoid formulations produced therewith show a particularly high storage stability (see Table).

The abovementioned dry powders are particularly suitable as addition to human foods and animal feeds and as addition to pharmaceutical preparations. Typical areas of use of the carotenoid-containing dry powders in the animal feeds sector are, for example, fish pigmentation in aquaculture, and egg yolk and broiler skin pigmentation in poultry rearing.

The procedure for the process of the invention is explained in detail in the following examples.

### EXAMPLE 1

Production of an Astaxanthin Dry Powder Using a Combination of Trehalose and Sodium Caseinate

66 g of crystalline astaxanthin and 15 g of α-tocopherol were suspended at a temperature of 30° C. in 496 g of an azeotropic isopropanol/water mixture at room temperature in a heatable receiver. The active substance suspension was then heated to 90° C. and mixed at a flow rate of 3.6 kg/h continuously with further isopropanol/water azeotrope at a temperature of 220° C. with a flow rate of 4.6 kg/h, the astaxanthin dissolving at a mixing temperature of 165° C. which was set up, under a pressure of 55 bar. This active substance solution was then immediately mixed with an aqueous phase consisting of a solution of 29 g of sodium caseinate and 166 g of trehalose in 8724 g of distilled water, in which the pH was adjusted to 9.5 with 1 M NaOH, at a flow rate of 55 kg/h.

The active substance particles produced on mixing had a particle size of 130 nm in the isopropanol/water mixture, with an E1/1 value¹) of 117.
1) The El/1 value defines in this connection the specific extinction of a 0.5% strength aqueous dispersion of a 20% by weight dry powder in a 1 cm cuvette at the absorption maximum.

The active substance suspension was then concentrated in a thin film evaporator to a concentration of about 27.4% dry content and spray dried. The dry powder had an astaxanthin content of 22.4% by weight. The dry powder redispersed in water had a particle size of 141 nm and an E1/1 value of 120.

### EXAMPLE 2

### COMPARATIVE EXAMPLE

Production of an Astaxanthin Dry Powder Using a Combination of Lactose and Sodium Caseinate

83.5 g of crystalline astaxanthin and 20 g of α-tocopherol were suspended at a temperature of 30° C. in 626 g of an azeotropic isopropanol/water mixture at room temperature in a heatable receiver. The active substance suspension was then heated to 90° C. and mixed at a flow rate of 2.1 kg/h continuously with further isopropanol/water azeotrope at a temperature of 220° C. with a flow rate of 2.6 kg/h, the astaxanthin dissolving at a mixing temperature of 165° C. which was set up, under a pressure of 55 bar. This active substance solution was then immediately mixed with an aqueous phase consisting of a solution of 83.5 g of sodium caseinate and 177 g of lactose in 20 580 g of distilled water, in which the pH was adjusted to 9.5 with 1 M NaOH, at a flow rate of 60 kg/h.

The active substance particles produced on mixing had a particle size of 133 nm in the isopropanol/water mixture, with an E1/1 value of 123.

The active substance suspension was then concentrated in a thin film evaporator to a concentration of about 6.9% dry content and spray dried. The dry powder had an astaxanthin content of 22.5% by weight. The dry powder redispersed in water had a particle size of 167 nm and an E1/1 value of 123.

### EXAMPLE 3

### COMPARATIVE EXAMPLE

Production of an Astaxanthin Dry Powder Using a Combination of Lactose and Soybean Protein

83.5 g of crystalline astaxanthin and 20 g of α-tocopherol were suspended at a temperature of 30° C. in 626 g of an azeotropic isopropanol/water mixture at room temperature in a heatable receiver. The active substance suspension was -then heated to 90° C. and mixed at a flow rate of 2.1 kg/h continuously with further isopropanol/water azeotrope at a temperature of 220° C. with a flow rate of 2.6 kg/h, the astaxanthin dissolving at a mixing temperature of 165° C. which was set up, under a pressure of 55 bar. This active substance solution was then immediately mixed with an aqueous phase consisting of a solution of 83.5 g of soybean protein and 177 g of lactose in 11 010 g of distilled water, in which the pH was adjusted to 9.5 with 1 M NaOH, at a flow rate of 32.5 kg/h.

The active substance particles produced on mixing had a particle size of 107 nm in the isopropanol/water mixture, with an E1/1 value of 124.

The active substance suspension was then concentrated in a thin film evaporator to a concentration of about 23.7% dry content and spray dried. The dry powder had an astaxanthin content of 23% by weight. The dry powder redispersed in water had a particle size of 317 nm and an E1/1 value of 101.

### EXAMPLE 4

### COMPARATIVE EXAMPLE

Production of an Astaxanthin Dry Powder Using a Combination of Dried Glucose Syrup (Glucidex® 47, from Roquette Freres) and Sodium Caseinate

66 g of crystalline astaxanthin and 15 g of α-tocopherol were suspended at a temperature of 30° C. in 496 g of an azeotropic isopropanol/water mixture at room temperature in a heatable receiver. The active substance suspension was then heated to 90° C. and mixed at a flow rate of 3.6 kg/h continuously with further isopropanol/water azeotrope at a temperature of 220° C. with a flow rate of 4.6 kg/h, the astaxanthin dissolving at a mixing temperature of 165° C. which was set up, under a pressure of 55 bar. This active substance solution was immediately thereafter mixed with an aqueous phase consisting of a solution of 28.7 g of sodium caseinate and 165.6 g of Glucidex 47 in 8724 g of distilled water, in which the pH was adjusted to 9.5 with 1 M NaOH, at a flow rate of 56 kg/h.

The active substance particles produced on mixing had a particle size of 144 nm in the isopronal/water mixture, with an E1/1 value of 115.

The active substance suspension was then concentrated in a thin film evaporator to a concentration of about 19.4% dry content and spray dried. The dry powder had an astaxanthin content of 23.6% by weight. The dry powder redispersed in water had a particle size of 623 nm and an E1/1 value of 119.

### EXAMPLE 5

### COMPARATIVE EXAMPLE

Production of an Astaxanthin Dry Powder Using a Combination of Dried Glucose Syrup (Glucidex® 47, from Roquette Freres) and Sodium Caseinate

83.5 g of crystalline astaxanthin and 20 g of α-tocopherol were suspended at a temperature of 30° C. in 626 g of an azeotropic isopropanol/water mixture at room temperature in a heatable receiver. The active substance suspension was then heated to ₉₀° C. and mixed at a flow rate of 3.6 kg/h continuously with further isopropanol/water azeotrope at a temperature of ₂₂0° C. with a flow rate of 4.6 kg/h, the astaxanthin dissolving at a mixing temperature of 165° C. which was set up, under a pressure of 55 bar. This active substance solution was then immediately mixed with an aqueous phase consisting of a solution of 83.5 g of sodium caseinate and 177 g of Glucidex 47 in 11 010 g of distilled water, in which the pH was adjusted to 9.5 with 1 M NaOH, at a flow rate of 56 kg/h.

The active substance particles produced on mixing had a particle size of 155 nm in the isopropanol/water mixture, with an E1/1 value of 116.

The active substance suspension was then concentrated in a thin film evaporator to a concentration of about 25% dry content and spray dried. The dry powder had an astaxanthin content of 22.3% by weight. The dry powder redispersed in water had a particle size of 179 nm and an E1/1 value of 117.

TABLE
Stability during storage of the astaxanthin dry
powders (thermal test at 60° C.)

| | | | | After 10 | | After 20 | |
|---|---|---|---|---|---|---|---|
| | | | | days | | days | |
| | | | Astaxanthin | Content | Loss | Content | Loss |
| Ex. | Sugar | Protein | content | (%) | (%) | (%) | (%) |
| 1 | Tre- | Sodium | 22.4 | 21.3 | 5.0 | 20.6 | 7.8 |
| | halose | caseinate | | | | | |
| 2 | Lactose | Sodium | 22.5 | 14.2 | 36.9 | 12.5 | 44.2 |
| | | caseinate | | | | | |
| 3 | Lactose | Soybean | 23.0 | 19.3 | 15.9 | 17.7 | 22.9 |
| | | protein | | | | | |
| 4 | Glucose | Sodium | 23.6 | 22.0 | 6.5 | 20.8 | 11.7 |
| | syrup | caseinate | | | | | |
| 5 | Glucose | Sodium | 22.3 | 18.3 | 17.8 | 16.6 | 25.6 |
| | syrup | caseinate | | | | | |

1. A process for producing dry powders of one or more carotenoids, which comprises
a) suspending one or more carotenoids in an aqueous molecular or colloidal solution of a mixture of trehalose and at least one protein-containing protective colloid and
b) converting the suspension which has formed into a dry powder by removing the water and, if appropriate, additionally used solvents and subsequent drying, if appropriate in the presence of a coating material.

2. The process according to claim 1, wherein the suspension prepared in process step a) is ground before conversion into a dry powder.

3. The process according to claim 1, wherein the suspension in stage a) comprises the following steps:
a₁) dissolving one or more carotenoids in a water-miscible organic solvent or in a mixture of water and a water-miscible organic solvent or
a₂) dissolving one or more carotenoids in a water-immiscible organic solvent and
a₃) mixing the solution obtained as in a,) or a₂) with an aqueous molecular or colloidal solution of a mixture of trehalose and at least one protein-containing protective colloid, resulting in the hydrophobic phase of the carotenoid as nanodisperse phase.

4. The process according to claim 1, wherein casein or a caseinate or mixtures thereof are used as protective colloid.

5. The process according to claim 1, wherein the carotenoids used are oxygen-containing carotenoids.

6. The process according to any of claim 5, wherein the oxygen-containing carotenoids are compounds selected from the group consisting of astaxanthin, canthaxanthin, lutein, zeaxanthin, citranaxanthin and ethyl β-apo-8′-carotenoate.

7. A process for producing an astaxanthin dry powder, wherein
a) astaxanthin is dissolved in a water-miscible organic solvent or a mixture of water and a water-miscible organic solvent at temperatures above 30° C.,
b) the resulting solution is mixed with an aqueous molecular or colloidal solution of a mixture of trehalose with casein or a caseinate or a mixture of trehalose with casein and a caseinate, and
c) the suspension which has formed is converted into a dry powder.

8. The process according to claim 7, wherein a mixture of trehalose and sodium caseinate is used as protective colloid in process step b).

9. A carotenoid-containing dry powder obtainable by a process as defined according to claim 1.

10. The dry powder according to claim 9 with a carotenoid content of from 0.1 to 40% by weight.

11. The dry powder according to claim 10, comprising 10 to 25% by weight of astaxanthin.

12. (canceled)

13. A human food which comprises the carotenoid-containing dry powders as defined according to claim 9.

14. A pharmaceutical which comprises the carotenoid-containing dry powders as defined according to claim 9.

15. An animal feed which comprises the carotenoid-containing dry powders as defined according to claim 9.
